(19) United States
(12) Patent Application Publication
(10) Pub. No.: US 2026/0294220 A1
(43) Pub. Date: Oct. 1, 2026

(54) ADAPTER

(71) Applicant: FUJIFILM Corporation, Tokyo (JP)

(72) Inventors: Yoshiyuki KUNUKI, Kanagawa (JP); Kazuyuki OGI, Kanagawa (JP)

(73) Assignee: FUJIFILM Corporation, Tokyo (JP)

(21) Appl. No.: 19/630,398

(22) Filed: Mar. 26, 2026

(30) Foreign Application Priority Data
Mar. 28, 2025 (JP) 2025-056267
Nov. 21, 2025 (JP) 2025-202229

(51) Int. Cl. A61B 1/00; A61B 1/015

(52) U.S. Cl. CPC A61B 1/00128; A61B 1/00119; A61B 1/015

(57) ABSTRACT

An adapter includes a first connection portion detachably connected to a control device that controls an endoscope, a second connection portion to which a first cord part is detachably connected to extend from an operation unit and including a liquid pipe line, and a housing that is provided with the first and second connection portions, the housing including a first surface that is on a side opposite to a first connection portion side in a movement direction of the housing when the first connection portion is connected to the control device and that intersects the movement direction, a second surface that is on a side opposite to the first connection portion side with respect to the first surface in the movement direction and that intersects the movement direction, and a third surface that connects the first and second surfaces. The second connection portion is provided on the third surface.

### CROSS-REFERENCE TO RELATED APPLICATIONS

This application is based upon and claims the benefit of priority from prior Japanese patent application No. 2025-56267, filed on Mar. 28, 2025 and Japanese patent application No. 2025-202229, filed on Nov. 21, 2025, the entire contents of which are incorporated herein by reference.

### BACKGROUND OF THE INVENTION

### Field of the Invention

The technology of the present disclosure relates to an adapter.

### Description of the Related Art

JP2024-68196A discloses an adapter for an endoscope system, the adapter comprising: a housing; a sensor that receives light waves emitted from a light source that can be connected to the adapter; and a converter that is connected to the sensor to convert a light intensity read from the sensor into a signal including a command for generating light using a light generator of an endoscope, in which the adapter is configured to output the signal to the endoscope.

JP2024-47524A discloses an endoscope connection mechanism that connects a light source device that supplies illumination light to an endoscope and a connector of the endoscope, the endoscope connection mechanism comprising: a connection fitting that has a hole into which a tubular portion of the connector is inserted and removed; and at least one optical shutter provided inside the hole, in which the tubular portion of the connection fitting accommodates a light guide member, the optical shutter includes a first portion, a second portion, and a third portion, the first portion has a first end that is a fixed end and a second end, the second portion has a third end that is in contact with the second end and a fourth end, the third portion has a fifth end that is in contact with the fourth end and a sixth end that is a free end, in a state where the tubular portion is not inserted into the hole, the third portion is positioned on an optical path of the illumination light, and in a state where the tubular portion is inserted into the hole, the first portion is pressed by the tubular portion and the third portion is positioned outside the optical path.

JP2023-87676A discloses a relay adapter that relays a processor and an insertion device, the relay adapter comprising: a first connector that is connected to the processor; a second connector that is connected to the insertion device; a power supply circuit that converts power supplied from the processor into power corresponding to the insertion device and that supplies the power to the insertion device; and a switching circuit that cuts off the supply of the power to the insertion device to the power supply circuit in a case where the first connector is connected to the processor and the second connector is not connected to the insertion device.

JP2020-62262A discloses a relay adapter that relays a processor and an endoscope device, the relay adapter comprising: a first connector portion that connects the endoscope device; a second connector portion that connects the processor; a storage unit that stores a program for controlling an operation of the relay adapter; and a controller that reads and executes the program from the storage unit, in which the controller executes processing of acquiring first specification information including a content of signal processing in the endoscope device and second specification information including a content of signal processing in the processor, processing of collating the first specification information with the second specification information and converting a signal received from the endoscope device such that the processor can process the signal to generate a converted signal based on a result of the collation, and processing of outputting the converted signal to the processor.

JP7177231B discloses a relay adapter that relays a processor and an endoscope device.

### SUMMARY OF THE INVENTION

An adapter according to one aspect of the technology of the present disclosure comprises a first connection portion that is attachably and detachably connected to a control device that controls an endoscope, a second connection portion to which a first cord part is attachably and detachably connected, the first cord part being provided to extend from an operation unit of the endoscope and including a liquid pipeline that communicates with a liquid discharge port provided on a distal end side of the endoscope, and a housing that is provided with the first connection portion and the second connection portion, the housing including a first surface that is on a side opposite to a first connection portion side in a movement direction of the housing in a case where the first connection portion is connected to the control device and that intersects the movement direction, a second surface that is on a side opposite to the first connection portion side with respect to the first surface in the movement direction and that intersects the movement direction, and a third surface that connects the first surface and the second surface, in which the second connection portion is provided on the third surface.

### BRIEF DESCRIPTION OF THE DRAWINGS

FIG. 1 is a schematic diagram showing a schematic configuration of an endoscope device 100 which is one aspect of the technology of the present disclosure.

FIG. 2 is a block diagram showing an internal configuration of a control device 80 and a reusable endoscope 10 connected to the control device 80.

FIG. 3 is a schematic diagram showing an example of an appearance of a connector 16A.

FIG. 4 is a front view of a connector connection portion 80A.

FIG. 5 is a schematic diagram showing an internal configuration of a single-use endoscope 40.

FIG. 6 is a perspective view showing an example of an external configuration of an adapter 50 and a connector 46A.

FIG. 7 is a side view of the adapter 50 shown in FIG. 6.

FIG. 8 is a perspective view of the connector 46A shown in FIG. 6 as viewed from a different direction.

FIG. 9 is a perspective view (part1) showing a state where the adapter 50 and the connector 46A shown in FIG. 6 are connected.

FIG. 10 is a perspective view (part 2) showing a state where the adapter 50 and the connector 46A shown in FIG. 6 are connected.

FIG. 11 is a block diagram showing an internal configuration in a state where the adapter 50 to which a connector 47A is connected is connected to the control device 80.

FIG. 12 is a schematic diagram for describing an image displayed on a display device 90.

FIG. 13 is a perspective view showing a modification example of the adapter 50.

FIG. 14 is a perspective view showing a configuration example of the connector 46A connected to the adapter 50 shown in FIG. 13.

FIG. 15 is a schematic diagram showing a modification example of the single-use endoscope 40.

### DESCRIPTION OF THE PREFERRED EMBODIMENTS

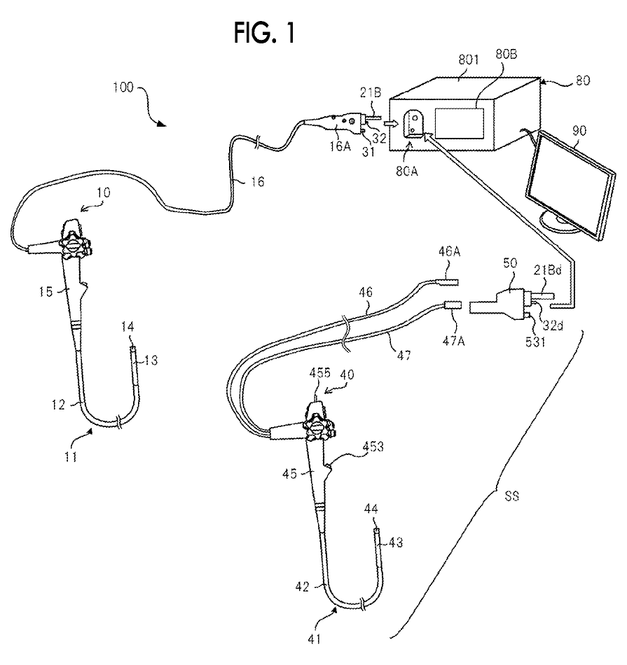

FIG. 1 is a schematic diagram showing a schematic configuration of an endoscope device 100 which is one aspect of the technology of the present disclosure. As shown in FIG. 1, the endoscope device 100 comprises a reusable endoscope 10 that can be repeatedly used by being reprocessed (disinfected and washed), a control device 80 that can control the reusable endoscope 10, a display device 90 such as a liquid-crystal display or an organic electro-luminescence (EL) display, and an endoscope system SS. The endoscope system SS comprises a single-use endoscope 40 that can be used once (one-time use), and an adapter 50 that relays a connection between the single-use endoscope 40 and the control device 80. In the endoscope device 100, components other than the endoscope system SS can use existing components. However, the control device 80 needs to update a program or the like.

The control device 80 includes an input device 80B (a physical button, a touch panel, a keyboard, a mouse, or the like) that receives an input from a user, and comprehensively controls the endoscope device 100 in response to an instruction input from the input device 80B.

The control device 80 is configured to be connected to the reusable endoscope 10 and to control the connected reusable endoscope 10. The control device 80 can be connected the single-use endoscope 40 via the adapter 50 and is configured to control the single-use endoscope 40 via the adapter 50.

The connection between the endoscope and the control device includes at least one of electrical connection between the endoscope and the control device (a state where signals or power can be transmitted and received by a wire, a radio wave, light, or the like), optical connection between the endoscope and the control device (a state where illumination light can be transmitted and received), or spatial connection between the endoscope and the control device (a state where a gas such as air can be transmitted and received).

The reusable endoscope 10 exemplifies a flexible endoscope, and comprises a flexible insertion part 11 that is inserted into a subject, an operation unit 15 that is provided at a base end portion of the insertion part 11 and that can operate the insertion part 11, a universal cord 16 that is provided to extend from the operation unit 15, and a connector 16A that is provided at an end part of the universal cord 16 and that is attachably and detachably connected to a connector connection portion 80A of the control device 80. The reusable endoscope 10 is not limited to the flexible endoscope and may be another type of endoscope such as a rigid endoscope.

The insertion part 11 includes, in order from a distal end side, a distal end part 14 formed of a rigid member, a bendable part 13 that is installed continuously on a base end side of the distal end part 14, and a soft part 12 that connects the bendable part 13 and the operation unit 15. The bendable part 13 is formed by connecting a plurality of bendable pieces (angle rings) and is configured to be bent in a predetermined direction in response to an operation of the operation unit 15. The soft part 12 is elongated and long and has flexibility.

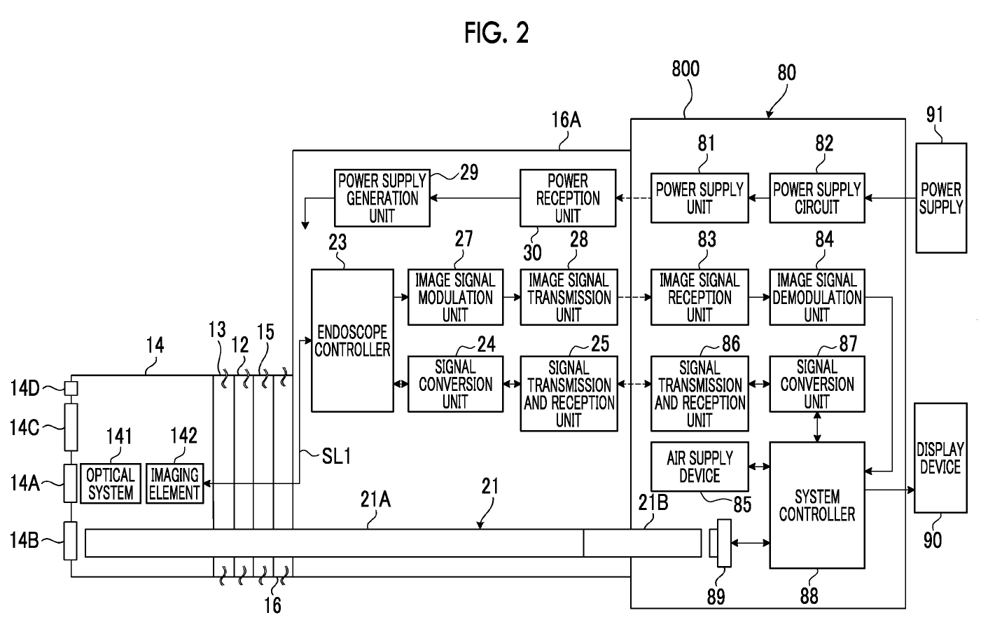

FIG. 2 is a block diagram showing an internal configuration of the control device 80 and the reusable endoscope 10 connected to the control device 80. As shown in FIG. 2, an outer surface of the distal end part 14 of the reusable endoscope 10 is provided with an observation window 14A that takes in subject light from a part to be observed, an illumination window 14B that emits illumination light for illuminating the part to be observed, a treatment tool outlet port 14C through which a treatment tool is led out, and an discharge port 14D that is an air/water supply nozzle that discharges a gas such as air or a liquid such as water.

An optical system 141 that forms an optical image of subject light from the part to be observed, which is taken in by the observation window 14A, and an imaging element 142 that converts the optical image formed by the optical system 141 into an image signal are provided inside the distal end part 14. The imaging element 142 is, for example, a charge-coupled device (CCD) image sensor, a complementary metal-oxide-semiconductor (CMOS) image sensor, or the like.

The image signal output from the imaging element 142 is transmitted to the connector 16A by a signal line SL1 that is inserted and disposed from the distal end of the insertion part 11 to the connector 16A through the inside of the insertion part 11, the operation unit 15, and the universal cord 16.

A light-emitting end of a light guide 21 that transmits the illumination light is disposed at a position facing the illumination window 14B inside the distal end part 14. The light guide 21 comprises an optical fiber bundle 21A that is inserted and disposed from the distal end part 14 to the connector 16A through the inside of the operation unit 15 and the universal cord 16, and a light guide rod 21B that is connected to a base end of the optical fiber bundle 21A. The light guide rod 21B is provided to protrude from the connector 16A to a side opposite to an operation unit 15 side. The light guide rod 21B is configured to allow the illumination light incident from a distal end of the light guide rod 21B to be incident on the optical fiber bundle 21A connected to a base end of the light guide rod 21B and is formed of a rigid member.

The operation unit 15 comprises an angle knob for adjusting an orientation of a distal end surface of the insertion part 11 in up-down and left-right directions, an air/water supply button for performing an operation of jetting air or a liquid from the discharge port 14D of the distal end part 14, and a release button for recording a captured image as a still image. An operation signal of a button or the like provided in the operation unit 15 is transmitted to the connector 16A by a signal line that is inserted and disposed from the inside of the operation unit 15 and the universal cord 16 to the connector 16A.

The universal cord 16 is covered with an outer wall part that is tubular and elongated and has flexibility. A signal line SL1 and the light guide 21 inserted and disposed in a cavity portion inside the insertion part 11 and inside the operation unit 15, and an air/water supply tube (not shown) or the like are inserted and disposed in a pipe inside the outer wall part.

The connector 16A is configured to be attached to and detached from the connector connection portion 80A provided in a housing 800 of the control device 80. By connecting the connector 16A to the connector connection portion 80A, power is supplied from the control device 80 to the reusable endoscope 10, an image signal is transmitted from the reusable endoscope 10 to the control device 80, and a control signal is transmitted and received between the reusable endoscope 10 and the control device 80 in a noncontact manner without connecting a physical wire.

As shown in FIG. 2, the connector 16A of the reusable endoscope 10 is provided with an endoscope controller 23 that controls each unit of the reusable endoscope 10, a signal conversion unit 24, a signal transmission and reception unit 25, an image signal modulation unit 27, an image signal transmission unit 28, a power supply generation unit 29, and a power reception unit 30. The endoscope controller 23 includes a processor that executes a program to perform various types of processing, and a memory. The endoscope controller 23 is connected to the imaging element 142 by the signal line SL1. An operation signal of various buttons (for example, the release button) included in the operation unit 15 is input to the endoscope controller 23.

The housing 800 of the control device 80 accommodates a power supply unit 81, a power supply circuit 82, an image signal reception unit 83, an image signal demodulation unit 84, an air supply device 85 that is configured of a pump that takes in and discharges air, a signal transmission and reception unit 86, a signal conversion unit 87, a system controller 88, and a light source device 89. The system controller 88 includes a processor that executes a program to perform various types of processing, and a memory.

The power required to drive an internal circuit of the reusable endoscope 10 is supplied from the control device 80 by noncontact power supply means consisting of the power supply unit 81 of the control device 80 and the power reception unit 30 of the reusable endoscope 10.

The noncontact power supply means is means for transmitting and receiving power in a noncontact manner by using electromagnetic coupling. In a case where the connector 16A is mounted on the connector connection portion 80A, the power supply unit 81 and the power reception unit 30 are disposed close to each other at a distance at which electromagnetic coupling is possible, and a state is set in which power transmission from the power supply unit 81 to the power reception unit 30 in a noncontact manner is possible. The power supply unit 81 is connected to a commercial power supply 91 outside the control device 80 via the power supply circuit 82. Power supplied from the commercial power supply 91 and generated by the power supply circuit 82 is supplied to the power supply unit 81. Power is supplied from the power supply unit 81 to the power reception unit 30 in a noncontact manner by the power supplied from the power supply circuit 82 to the power supply unit 81. The power reception unit 30 receives power from the power supply unit 81 in a noncontact manner.

It is preferable that the power supply unit 81 is a primary coil connected to the power supply circuit 82 and that the power reception unit 30 is a secondary coil electromagnetically coupled to the primary coil. Examples of the structure of the primary coil and the secondary coil include a structure having a substrate having a flat surface and a coil spirally wound on the flat surface.

The power supply generation unit 29 of the connector 16A generates a power supply voltage to be supplied to each unit of the reusable endoscope 10 from the power received by the power reception unit 30. The image signal output from the imaging element 142 of the reusable endoscope 10 is transmitted to the endoscope controller 23 by the signal line SL1. The endoscope controller 23 performs necessary processing such as amplification processing on the image signal. The processing performed on the image signal by the endoscope controller 23 may not include demosaicing processing (processing of causing a plurality of pieces of color information to be included in one pixel of the image signal), gamma-correction processing, white balance adjustment processing, and defect correction processing (processing of interpolating a signal of a defective pixel of the imaging element 142 included in the image signal from the periphery). As a result, the manufacturing cost of the reusable endoscope 10 can be reduced.

The image signal modulation unit 27 performs optical modulation based on the image signal to generate an image light signal in order to transmit the image signal processed by the endoscope controller 23 to the control device 80 by optical communication.

The image signal transmission unit 28 irradiates the control device 80 with light (light based on the image signal) in accordance with the image light signal generated by the image signal modulation unit 27. The image signal transmission unit 28 may be a light-emitting device that can emit light for optical communication, and examples thereof include a laser light-emitting element and a light-emitting diode. The laser light-emitting element refers to an element that emits laser light, which is coherent light, and examples thereof include a gas laser, a solid-state laser, and a semiconductor laser.

The signal conversion unit 24 performs optical modulation based on the control signal and demodulation of the control signal in order to transmit and receive the control signal that needs to be transmitted and received between the reusable endoscope 10 and the control device 80 via optical communication. The control signal may include a first control signal transmitted from the control device 80 to the reusable endoscope 10 to control the imaging element 142 and the like, and a second control signal transmitted from the reusable endoscope 10 to the control device 80.

The signal conversion unit 24 performs optical modulation based on the second control signal output from the endoscope controller 23 to generate a second control light signal. The signal conversion unit 24 demodulates an electric signal output from the signal transmission and reception unit 25 to obtain the first control signal.

The signal transmission and reception unit 25 includes a light-emitting device that irradiates the control device 80 with light (light based on the second control signal) in accordance with the second control light signal generated by the signal conversion unit 24, and a light-receiving device that receives light (light based on the first control signal) in accordance with a first control light signal modulated based on the first control signal generated by the control device 80.

Examples of the light-emitting device of the signal transmission and reception unit 25 include a laser light-emitting element and a light-emitting diode. Examples of the light-receiving device of the signal transmission and reception unit 25 include a light-receiving element such as a semiconductor device such as a photodiode or a phototransistor.

The image signal reception unit 83 of the control device 80 is a light-receiving device that receives light emitted from the image signal transmission unit 28 and that converts the light into an electric signal, and examples thereof include a light-receiving element such as a semiconductor device such as a photodiode or a phototransistor.

The image signal demodulation unit 84 demodulates the image signal transmitted from the reusable endoscope 10 based on the electric signal output from the image signal reception unit 83. The image signal is processed by the system controller 88 to generate an endoscopic image, and the endoscopic image is displayed on the display device 90.

The signal conversion unit 87 performs optical modulation based on the first control signal output from the system controller 88 to generate the first control light signal. The signal conversion unit 87 demodulates the electric signal output from the signal transmission and reception unit 86 to obtain the second control signal.

The signal transmission and reception unit 86 includes a light-emitting device that irradiates the light-receiving device of the signal transmission and reception unit 25 of the reusable endoscope 10 with light (light based on the first control signal) in accordance with the first control light signal generated by the signal conversion unit 87, and a light-receiving device that receives the light emitted from the light-emitting device of the signal transmission and reception unit 25 of the reusable endoscope 10.

Examples of the light-emitting device of the signal transmission and reception unit 86 include a laser light-emitting element and a light-emitting diode. Examples of the light-receiving device of the signal transmission and reception unit 86 include a light-receiving element such as a semiconductor device such as a photodiode or a phototransistor.

In a case where the connector 16A is connected to the connector connection portion 80A, the image signal transmission unit 28 and the image signal reception unit 83 are disposed close to each other at a distance at which optical communication can be performed, and a state is set in which optical communication can be performed from the image signal transmission unit 28 to the image signal reception unit 83 in a noncontact manner. In addition, the signal transmission and reception unit 25 and the signal transmission and reception unit 86 are disposed close to each other at a distance at which optical communication can be performed, and a state is set in which optical communication can be performed between the signal transmission and reception unit 25 and the signal transmission and reception unit 86 in a noncontact manner. As described above, in the present embodiment, the communication of the image signal and the control signal between the reusable endoscope 10 and the control device 80 is performed by the noncontact optical communication means. A method of optical communication is not particularly limited, and various methods can be employed. Instead of the optical communication, wired communication, wireless communication, or magnetic communication can also be used.

The light source device 89 has, for example, a light source including a xenon lamp, or a semiconductor device such as a laser diode or a light-emitting diode. In a case where the connector 16A is connected to the connector connection portion 80A of the control device 80, the light guide rod 21B of the reusable endoscope 10 is inserted into the housing 800 of the control device 80, and the light-emitting unit of the light source device 89 accommodated in the housing 800 and the light guide rod 21B are aligned. As a result, the illumination light from the light source device 89 can be transmitted to the distal end part 14 through the light guide 21.

The light source device 89 is controlled by the system controller 88. The system controller 88 may be divided into a processor that controls the light source device 89 and a processor that performs processing other than the control of the light source device 89. The control device 80 may have a configuration in which a housing that accommodates the light source device 89 and a processor that controls the light source device 89 and a housing that accommodates blocks other than the light source device 89 and a processor that controls the blocks are separated from each other, and the two processors are electrically connected to each other by a cable or the like.

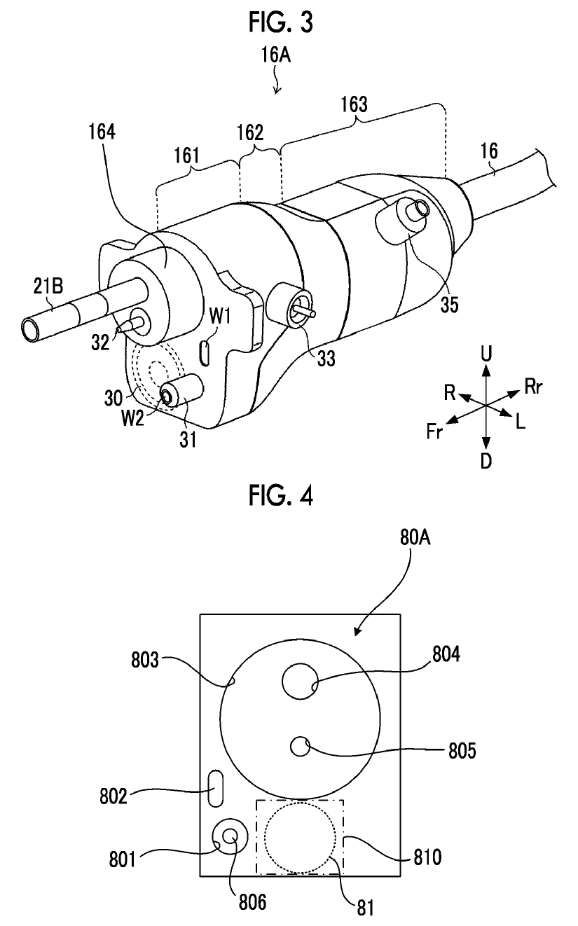

FIG. 3 is a schematic diagram showing an example of an appearance of the connector 16A. In FIG. 3, a direction from an end of the connector 16A on the operation unit 15 side to an end of the connector 16A on a side opposite to the operation unit 15 side is shown as a front direction Fr, and a direction opposite to the front direction Fr is shown as a rear direction Rr. The front direction Fr and the rear direction Rr are collectively referred to as a front-rear direction. The front-rear direction is a longitudinal direction of the connector 16A. In FIG. 3, a left-right direction (right direction R and left direction L) and an up-down direction (upward direction U and downward direction D) that are perpendicular to each other are shown as two directions perpendicular to the front-rear direction. FIG. 4 is a front view of the connector connection portion 80A.

As shown in FIG. 3, the housing of the connector 16A is composed of a first connector case 161, a second connector case 162 on a rear side of the first connector case 161, and a third connector case 163 on a rear side of the second connector case 162.

A cylindrical engagement protrusion 164 is provided to protrude in the front direction Fr on a connection surface of the first connector case 161 with the connector connection portion 80A. The light guide rod 21B protrudes in the front direction Fr from a distal end surface of the engagement protrusion 164. An air supply fitting 32 is provided to protrude in the front direction Fr below the light guide rod 21B on the distal end surface of the engagement protrusion 164. The air supply fitting 32 communicates with an air supply pipeline that is disposed inside the reusable endoscope 10 to supply air to the distal end part 14 of the reusable endoscope 10.

As shown in FIG. 4, the housing 800 of the control device 80 is provided with a cylindrical first engagement recess 803, an insertion hole 804 that penetrates from a bottom surface of the first engagement recess 803 into the housing 800, an air supply hole 805 that penetrates from the bottom surface of the first engagement recess 803 into the housing 800, a window 802 for optical communication, a second engagement recess 801 having a window 806 for optical communication on a bottom surface, and a power supply region 810 in which a coil constituting the power supply unit 81 is disposed in close proximity, and the connector connection portion 80A is configured by these units.

An image signal transmission connector 31 shown in FIG. 3 is used for aligning the image signal transmission unit 28 of the reusable endoscope 10 and the image signal reception unit 83 of the control device 80. In particular, the image signal transmission unit 28 is disposed in an extension direction of a central axis of the image signal transmission connector 31. A window W2 is provided at a distal end of the image signal transmission connector 31 in order to transmit light. The image signal transmission connector 31 is inserted into the second engagement recess 801 of the housing 800, and the window W2 is disposed to face the window 806 of the housing 800. Light passes through the windows, and transmission and reception of light based on the image signal is performed between the image signal transmission unit 28 and the image signal reception unit 83.

In a case where the connector 16A is connected to the connector connection portion 80A, since the engagement protrusion 164 is engaged with the first engagement recess 803 in a state where the light guide rod 21B is inserted into the insertion hole 804 and the air supply fitting 32 is inserted into the air supply hole 805, the connector 16A is stably held by the control device 80. In addition, the light guide rod 21B and the light source device 89 are positioned, so that the illumination light can be supplied from the light source device 89 to the reusable endoscope 10. In addition, the air supply fitting 32 is connected to the air supply device 85, so that the air can be supplied from the air supply device 85 to the air supply pipeline of the reusable endoscope 10. The air supply hole 805 constitutes a fluid discharge port that discharges a gas (air) as a fluid. In addition, the image signal transmission connector 31 is engaged with the second engagement recess 801, the window 806 and the window W2 are disposed to face each other, and the optical communication is possible.

As shown in FIG. 3, a window W1 is provided at a position corresponding to the signal transmission and reception unit 25 on the connection surface of the first connector case 161 with the connector connection portion 80A. The window 802 and the window W1 provided in the control device 80 are disposed to face each other, and transmission and reception of light based on the control signal is performed between the signal transmission and reception unit 25 and the signal transmission and reception unit 86 through the window 802 and the window W1.

The power reception unit 30 is disposed inside the first connector case 161 at a position close to the connection surface of the first connector case 161 with the connector connection portion 80A. Since the power reception unit 30 is disposed inside the first connector case 161, the power reception unit 30 is not exposed to the outside.

A water supply connection fitting 33 is provided on a side surface of the first connector case 161. The water supply connection fitting 33 is connected to a water supply tank (not shown). By operating the air/water supply button of the operation unit 15, air or water can be supplied to the distal end part 14. The dirt on the observation window 14A of the distal end part 14 is removed by water supplied from the water supply tank connected to the water supply connection fitting 33 and jetted from the discharge port 14D of the distal end part 14. In addition, the water droplets on the observation window 14A are removed, or a lumen of the subject is expanded by air supplied from the air supply device 85 of the control device 80 and jetted from the discharge port 14D of the distal end part 14.

A ventilation connector 35 is provided on a side surface of the third connector case 163. The ventilation connector 35 enables an airtightness test for examining airtightness failure of the insertion part 11. The ventilation connector 35 communicates with the inside of the connector 16A. Since the inside of the connector 16A communicates with the inside of each of the universal cord 16, the operation unit 15, and the insertion part 11, the ventilation connector 35 communicates with the inside of the insertion part 11.

The universal cord 16 protrudes from the third connector case 163.

The system controller 88 of the control device 80 controls each unit of the control device 80, and in a case where the connector 16A is connected to the connector connection portion 80A to connect the reusable endoscope 10 to the control device 80, the system controller 88 transmits a control signal (including a signal for designating the sensitivity and the exposure time of the imaging element 142) to the endoscope controller 23 of the reusable endoscope 10 to control the reusable endoscope 10. In addition, in a case where the reusable endoscope 10 is connected to the control device 80, the system controller 88 performs control of generating an endoscopic image by performing demosaicing processing, gamma-correction processing, white balance adjustment processing, defect correction processing, and the like on the image signal (first image signal) received from the reusable endoscope 10 and storing the endoscopic image in a storage medium, and control of displaying the endoscopic image on the display device 90.

In a case where the reusable endoscope 10 is connected to the control device 80, the system controller 88 controls the light source device 89 to control the amount of light emitted from the illumination window 14B of the distal end part 14 through the light guide 21. The user operates the input device 80B to input a setting instruction of a first target value, which is a target value of the brightness of the image signal obtained by imaging with the reusable endoscope 10, to the system controller 88. The system controller 88 performs control of setting the target value of the brightness of the image signal to the first target value in response to the setting instruction and supplying light having an amount of light based on the first target value from the light source device 89 to the reusable endoscope 10 (light guide 21). The amount of light based on the first target value is determined to a value such that the brightness of the image signal received from the reusable endoscope 10 converges to the first target value by comparing the brightness with the first target value.

In a case where the reusable endoscope 10 is connected to the control device 80, the system controller 88 enables a first light amount limiting mode and a second light amount limiting mode for limiting the amount of illumination light emitted from the reusable endoscope 10, and a light transmission mode for setting the amount of illumination light emitted from the reusable endoscope 10 to an amount that can be transmitted from an inside of the subject to an outside of the subject, as operation modes of the light source device 89. The enabling of the mode means that the mode can be set by the operation of the user.

Since the reusable endoscope 10 emits the illumination light supplied from the light source device 89 from the distal end part 14, the amount of the illumination light can be increased. Therefore, the amount of heat generated in the distal end part 14 may increase. The first light amount limiting mode is a mode in which an upper limit is set on the amount of illumination light in order to suppress the heat generation of the distal end part 14 in the subject.

In a state where the insertion part 11 of the reusable endoscope 10 is outside the subject, the reusable endoscope 10 is present in an examination room in which the illumination is dimmed. In this state, since the difference between the brightness of the image signal and the first target value is larger than in a case where the distal end part 14 is inside the subject, the state where the amount of illumination light is large continues. In a case where the state where the amount of illumination light is large continues for a long time, the distal end part 14 generates heat, and may be affected in a case of being inserted into the subject. The second light amount limiting mode is a mode in which the amount of illumination light is reduced to be lower than the amount of light based on the first target value in a case where the cumulative amount of illumination light from the start of the emission of the illumination light is equal to or higher than a predetermined value, to suppress the heat generation. In a state where the insertion part 11 is inside the subject, since the state where the amount of illumination light is large is unlikely to continue, the second light amount limiting mode may be released.

The light transmission mode is a mode in which light having an amount of light that can be transmitted through the subject is intermittently emitted from the illumination window 14B such that an operator can check the position of the distal end part 14 in the subject.

As shown in FIG. 1, the single-use endoscope 40 exemplifies a flexible endoscope, and comprises a flexible insertion part 41 that is inserted into a subject, an operation unit 45 that is provided at a base end portion of the insertion part 41 and that can operate the insertion part 41, a first cord part 46 that is provided to extend from the operation unit 45, a connector 46A that is provided at an end part of the first cord part 46 and that is attachably and detachably connected to the adapter 50, a second cord part 47 that is provided to extend from the operation unit 45, and a connector 47A that is provided at an end part of the second cord part 47 and that is attachably and detachably connected to the adapter 50. The connector 46A is configured to be electrically unconnectable to the adapter 50. The connector 47A is configured to be electrically connected to the adapter 50.

The single-use endoscope 40 is not limited to the flexible endoscope, and may be another type of endoscope such as a rigid endoscope. Each of the first cord part 46 and the second cord part 47 is configured of a tubular outer sheath and a member that is inserted and disposed inside the outer sheath.

The insertion part 41 includes, in order from a distal end side, a distal end part 44 formed of a rigid member, a bendable part 43 that is installed continuously on a base end side of the distal end part 44, and a soft part 42 that connects the bendable part 43 and the operation unit 45. The bendable part 43 is formed by connecting a plurality of bendable pieces (angle rings) and is configured to be bent in a predetermined direction in response to an operation of the operation unit 45. The soft part 42 is elongated and long and has flexibility.

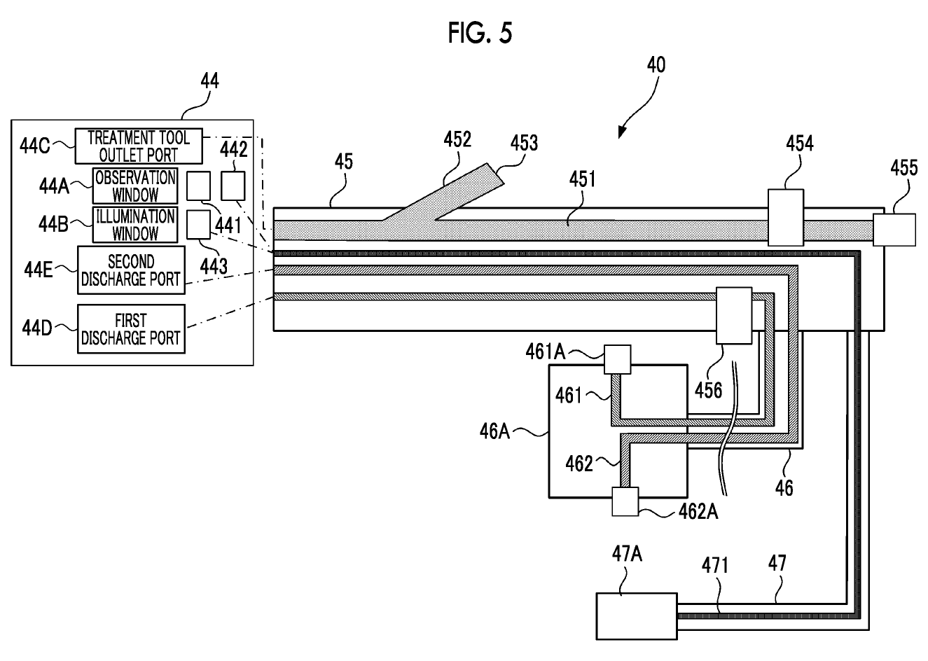

FIG. 5 is a schematic diagram showing an internal configuration of the single-use endoscope 40. As shown in FIG. 5, an outer surface of the distal end part 44 of the single-use endoscope 40 is provided with an observation window 44A that takes in subject light from a part to be observed, an illumination window 44B that emits illumination light for illuminating the part to be observed, a treatment tool outlet port 44C through which a treatment tool is led out, a first discharge port 44D that discharges a fluid, and a second discharge port 44E that discharges a fluid. The first discharge port 44D is an air/water supply nozzle. The second discharge port 44E is a secondary water supply port (water jet port).

The first discharge port 44D is configured to discharge any of air (gas) or water (liquid) toward the observation window 44A. The observation window 44A can be washed with the water discharged from the first discharge port 44D. The water droplets adhered to the observation window 44A can be removed by the air discharged from the first discharge port 44D. The second discharge port 44E is configured to supply water (liquid) toward a lumen of the subject, and the dirt in the lumen can be removed by the water supply.

The connector 46A of the first cord part 46 is provided with an air/water supply connection fitting 461A to which an air/water supply device can be connected, and a secondary water supply fitting 462A to which a syringe that discharges a liquid can be connected.

An air/water supply pipeline 461 as a liquid pipeline and a gas pipeline that communicates with the first discharge port 44D is provided inside the insertion part 41. The air/water supply pipeline 461 is composed of, for example, a rigid pipe provided at the distal end part 44 of the insertion part 41 and a flexible tube connected to the rigid pipe. The air/water supply pipeline 461 is connected to the air/water supply connection fitting 461A of the connector 46A from the first discharge port 44D through the inside of the insertion part 41, the operation unit 45, and the first cord part 46.

In a case where the air/water supply device connected to the air/water supply connection fitting 461A is operated, air or water can be supplied to the first discharge port 44D through the air/water supply pipeline 461. A valve mechanism 456 is provided inside the operation unit 45 at a middle of the air/water supply pipeline 461. The valve mechanism 456 is configured as, for example, an air/water supply button. By opening and closing a valve provided in the valve mechanism 456, the supply of the air or the water from the air/water supply device connected to the air/water supply connection fitting 461A to the first discharge port 44D can be switched on and off. The air/water supply pipeline 461 constitutes a first pipeline through which a liquid for washing the observation window 44A passes.

A water supply pipeline 462 as a liquid pipeline that communicates with the second discharge port 44E is provided inside the insertion part 41. The water supply pipeline 462 is composed of, for example, a rigid pipe provided at the distal end part 44 of the insertion part 41 and a flexible tube connected to the rigid pipe. The water supply pipeline 462 is connected to the secondary water supply fitting 462A of the connector 46A from the second discharge port 44E through the inside of the insertion part 41, the operation unit 45, and the first cord part 46. In a case where the syringe connected to the secondary water supply fitting 462A is operated, the water accommodated in the syringe can be supplied to the second discharge port 44E through the water supply pipeline 462. The water supply pipeline 462 constitutes a second pipeline through which a liquid for liquid supply to the subject passes.

A treatment tool pipeline 451 that communicates with the treatment tool outlet port 44C is provided inside the insertion part 41. The treatment tool pipeline 451 has a branch path 452 inside the operation unit 45, and an outlet of the branch path 452 is a treatment tool inlet port 453 of the operation unit 45.

A suction nozzle 455 is connected to a base end side (a side opposite to the treatment tool outlet port 44C) of the treatment tool pipeline 451 with respect to the branch path 452. The suction nozzle 455 protrudes from a housing of the operation unit 45 and can be connected to a suction device (not shown). A valve mechanism 454 is provided between the branch path 452 of the treatment tool pipeline 451 and the suction nozzle 455. The valve mechanism 454 is configured as, for example, a suction button.

By opening and closing a valve provided in the valve mechanism 454, a negative pressure can be generated by suctioning air inside the treatment tool pipeline 451 from the suction device connected to the suction nozzle 455. As a result, a body fluid or the like inside the subject can be suctioned from the treatment tool outlet port 44C via the treatment tool pipeline 451. As described above, the treatment tool outlet port 44C functions as a suction port provided on the distal end side of the insertion part 41, and the treatment tool pipeline 451 functions as a suction pipeline that is provided to extend from the suction port to the operation unit 45.

An optical system 441 that forms an optical image of subject light from the part to be observed, which is taken in by the observation window 44A, an imaging element 442 that converts the optical image formed by the optical system 441 into an image signal, and a light source 443 that generates illumination light emitted from the illumination window 44B are provided at the distal end part 44 of the insertion part 41. The light source 443 includes a light-emitting element composed of a semiconductor device such as a light-emitting diode. By using the semiconductor device as the light-emitting element included in the light source 443, the manufacturing cost of the single-use endoscope 40 can be reduced.

A signal line 471 connected to the imaging element 442 and the light source 443 is provided inside the insertion part 41. The signal line 471 is connected to the connector 47A from the distal end part 44 through the inside of the insertion part 41, the operation unit 45, and the second cord part 47.

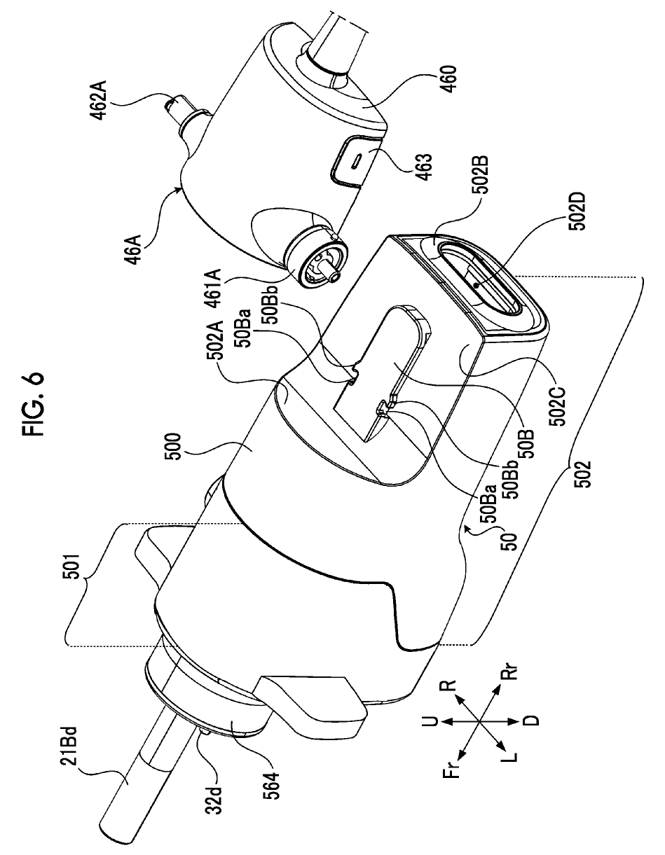

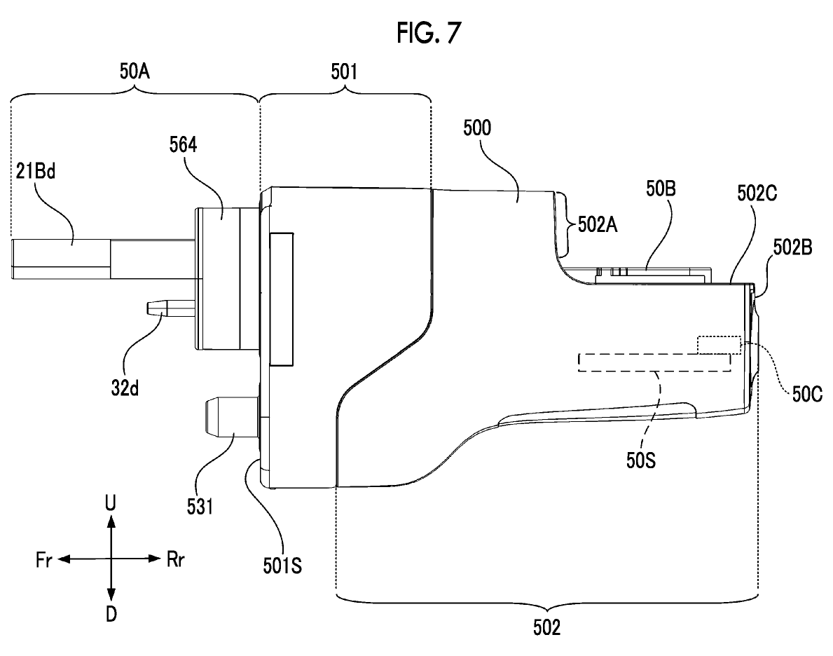

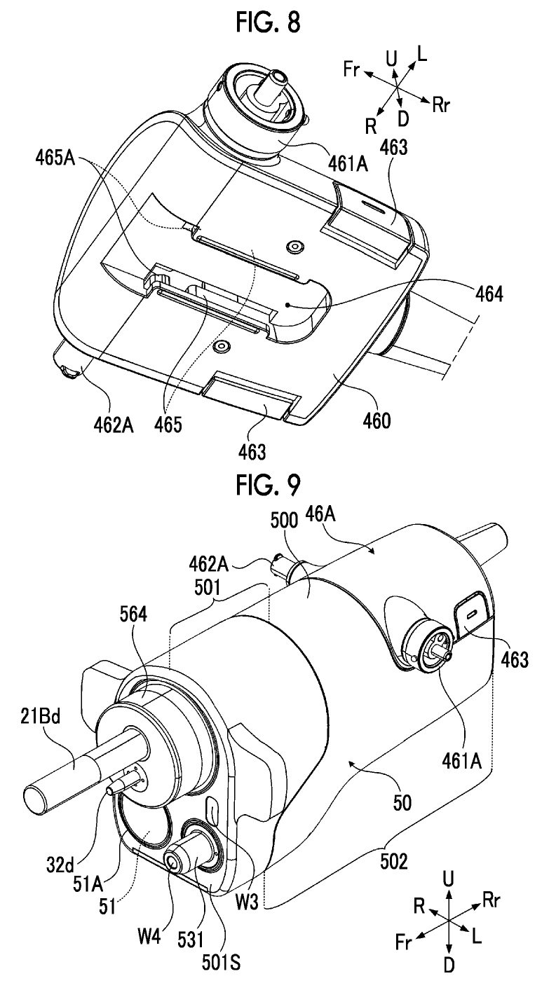

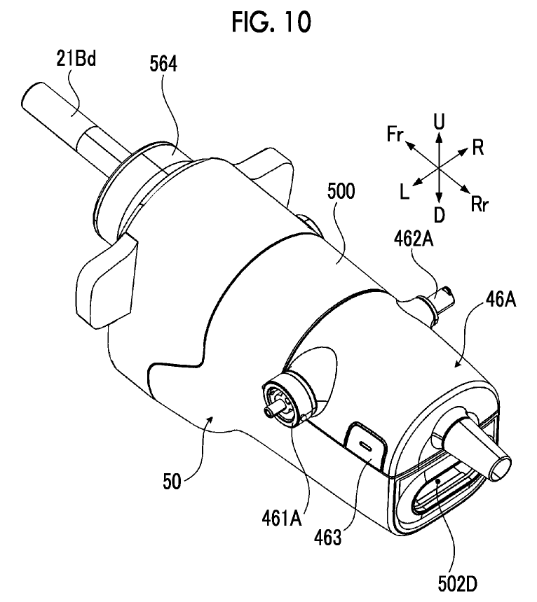

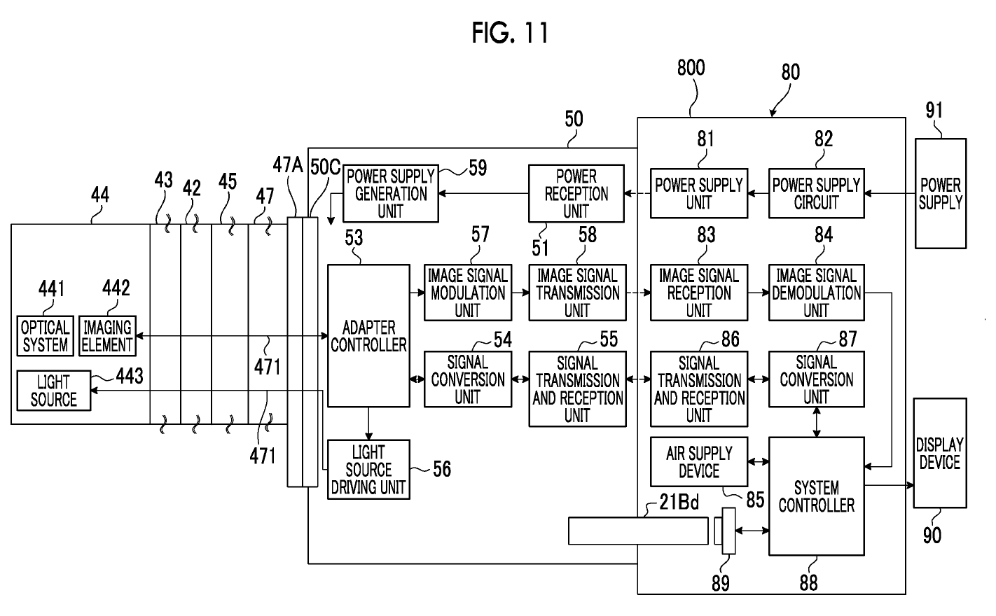

FIG. 6 is a perspective view showing an example of an external configuration of the adapter 50 and the connector 46A. FIG. 7 is a side view of the adapter 50 shown in FIG. 6. FIG. 8 is a perspective view of the connector 46A shown in FIG. 6 as viewed from a different direction. FIGS. 9 and 10 are perspective views showing a state where the adapter 50 and the connector 46A shown in FIG. 6 are connected. In FIGS. 6 - 10, a direction in which the adapter 50 is moved in a case where the adapter 50 is connected to the connector connection portion 80A of the control device 80 is shown as a front-rear direction (a direction in which the adapter 50 is brought close to the control device 80 is the front direction Fr, and a direction in which the adapter 50 is moved away from the control device 80 is the rear direction Rr). In FIGS. 6 -10, two directions (left-right direction and up-down direction) perpendicular to the front-rear direction are shown in the same manner as in FIG. 3.

As shown in FIGS. 6 and 7, a housing 500 of the adapter 50 is composed of a first adapter case 501 having substantially the same shape as the first connector case 161 of the connector 16A and a second adapter case 502 that is installed continuously behind the first adapter case 501.

As shown in FIG. 7, a first connection portion 50A that is attachably and detachably connected to the connector connection portion 80A is provided on a surface 501S of the first adapter case 501 on a front side. The first connection portion 50A is configured of an engagement protrusion 564 having the same shape as the engagement protrusion 164 of the connector 16A, a dummy rod 21Bd that is provided to protrude from a distal end surface of the engagement protrusion 564 and that has the same shape as the light guide rod 21B of the connector 16A, a dummy fitting 32d that is provided to protrude from the distal end surface of the engagement protrusion 564 and that has the same shape as the air supply fitting 32 of the connector 16A, and an image signal transmission connector 531 that is provided to protrude from the surface 501S and that has the same shape as the image signal transmission connector 31 of the connector 16A. A window W4 (see FIG. 9) having the same configuration as the window W2 of the connector 16A is provided at a distal end of the image signal transmission connector 531. The first connection portion 50A constitutes a device-side connection portion that is attachably and detachably connected to the control device 80. The dummy rod 21Bd constitutes an insertion member that is insertable into the insertion hole 804 of the connector connection portion 80A.

As shown in FIG. 9, a window W3 having the same configuration as the window W1 of the connector 16A is provided on the surface 501S of the first adapter case 501. In addition, the surface 501S includes a power reception region 51A in which a power reception unit 51 (described below) accommodated inside the housing 500 is disposed in close proximity.

In a case where the adapter 50 is connected to the connector connection portion 80A, since the engagement protrusion 564 is engaged with the first engagement recess 803 in a state where the dummy rod 21Bd is inserted into the insertion hole 804 and the dummy fitting 32d is inserted into the air supply hole 805, the adapter 50 is stably held by the control device 80. The dummy rod 21Bd is a substantially cylindrical member having a closed distal end surface. Therefore, in a state where the dummy rod 21Bd is inserted into the insertion hole 804, the light-emitting port of the light source device 89 accommodated in the housing 800 of the control device 80 is shielded by the dummy rod 21Bd. The dummy rod 21Bd also functions as a guide in a case where the adapter 50 is connected to the control device 80. In a case where the dummy rod 21Bd is inserted into the insertion hole 804, the control device 80 can detect the dummy rod 21Bd and detect that the adapter 50 is connected to the control device 80. The dummy fitting 32d is a substantially cylindrical member having a closed distal end surface. Therefore, in a state where the dummy fitting 32d is inserted into the air supply hole 805, an air discharge port of the air supply device 85 accommodated in the housing 800 of the control device 80 is closed by the dummy fitting 32d. The dummy fitting 32d is not essential and may be omitted.

In a case where the adapter 50 is connected to the connector connection portion 80A, since the window 802 of the control device 80 and the window W3 of the adapter 50 are disposed to face each other, and the window 806 of the control device 80 and the window W4 of the adapter 50 are disposed to face each other, a state is set in which the optical communication can be performed.

As shown in FIG. 7, the second adapter case 502 has a first surface 502A (in the example of FIG. 7, a surface including a region orthogonal to the front-rear direction) that is on a side opposite to the first connection portion 50A in the front-rear direction and that intersects the front-rear direction, a second surface 502B (in the example of FIG. 7, a surface including a region orthogonal to the front-rear direction) that is on a side (rear side) opposite to the first connection portion 50A with respect to the first surface 502A in the front-rear direction and that intersects the front-rear direction, and a third surface 502C that connects the first surface 502A and the second surface 502B. The second surface 502B constitutes an end surface (end surface on a side opposite to a side on which the first connection portion 50A is provided) of the adapter 50 on a rear side.

The first surface 502A and the second surface 502B are provided at positions shifted in the up-down direction (first direction) as viewed in the front-rear direction. The first surface 502A is disposed above the second surface 502B. A space is generated between a position of an upper end of the first surface 502A and a position of an upper end of the second surface 502B, and the connector 46A of the first cord part 46 can be disposed in the space.

The third surface 502C is a surface including a region intersecting the up-down direction, and is a plane orthogonal to the up-down direction in the example of FIG. 7. In the second adapter case 502 between the position of the first surface 502A and the position of the second surface 502B in the front-rear direction, a circuit board 50S on which electronic components mounted on the adapter 50 are mounted is provided. A substrate connector 50C for connecting to the connector 47A of the second cord part 47 is provided on the circuit board 50S.

As shown in FIG. 6, an opening 502D is provided in the second surface 502B, and the substrate connector 50C is exposed inside the opening 502D. In a case where the connector 47A of the second cord part 47 is inserted into the opening 502D, the connector 47A and the substrate connector 50C are electrically connected. The opening 502D and the substrate connector 50C exposed inside the opening 502D constitute a third connection portion or a first endoscope-side connection portion to which the second cord part 47 is attachably and detachably connected.

An engagement protrusion 50B to which the connector 46A of the first cord part 46 is attachably and detachably connected is provided on the third surface 502C. The engagement protrusion 50B is composed of a substantially flat plate-shaped protrusion that protrudes in the upward direction U from the third surface 502C and that extends in the front-rear direction. The engagement protrusion 50B constitutes a second connection portion or a second endoscope-side connection portion to which the first cord part 46 is attachably and detachably connected.

As shown in FIG. 8, an engagement recess 464 that is engageable with the engagement protrusion 50B of the adapter 50 is provided in a housing 460 of the connector 46A. The engagement recess 464 has a substantially rectangular parallelepiped shape that is open on the front side and extends in the front-rear direction. A pair of engagement members 465 that are movable in the left-right direction are provided inside left and right wall surfaces of the engagement recess 464 in the housing 460. As shown in FIG. 6, the engagement protrusion 50B of the adapter 50 is provided with a recess 50Ba that is engageable with the pair of engagement members 465.

A pair of interlocking members 463, which are movable left and right and interlock with the engagement member 465, are provided on left and right rear side surfaces of the housing 460 of the connector 46A. In a state where the pair of interlocking members 463 are not pressed, a front end 465A (see FIG. 8) of the pair of engagement members 465 is in a state of being entered into the engagement recess 464. In this state, in a case where the engagement recess 464 of the connector 46A is fitted to the rear side of the engagement protrusion 50B from the front side and the connector 46A is slid in the front direction Fr, the front ends 465A of the pair of engagement members 465 of the connector 46A move forward while sliding on left and right side surfaces of the engagement protrusion 50B, and engage with the recess 50Ba by passing over a restricting protrusion 50Bb (see FIG. 6) provided on the engagement protrusion 50B. In a state where the front ends 465A are engaged with the recess 50Ba, even in a case where the connector 46A is moved in the rear direction Rr, since the movement of the front ends 465A is restricted by the restricting protrusion 50Bb, the connector 46A cannot be removed. As described above, by sliding the housing 460 of the connector 46A in the front direction Fr with respect to the adapter 50, the connector 46A can be connected to the adapter 50 as shown in FIGS. 9 and 10.

A direction (front direction Fr) in which the adapter 50 is moved in a case where the adapter 50 is connected to the connector connection portion 80A and a direction in which the connector 46A and the connector 47A are moved in a case where the connector 46A and the connector 47A are connected to the adapter 50 are the same. Therefore, a series of operations of connecting the connector 46A and the connector 47A to the adapter 50 and then connecting the adapter 50 to the connector connection portion 80A can be smoothly performed, and the efficiency of the endoscopy can be improved.

In addition, in the present embodiment, the second endoscope-side connection portion connected to the connector 46A and the first endoscope-side connection portion connected to the connector 47A are disposed on a side opposite to the first connection portion 50A with respect to a control device 80 side in a case where the first connection portion 50A is connected to the control device 80. Therefore, the adapter 50 can be used to be connected to the control device 80, and then the first cord part 46 and the second cord part 47 can be connected to the adapter 50. Even in such a case, the adapter 50 and the connector can be easily connected to each other.

A notch corresponding to an outer shape of the connector 46A is provided on a rear side and an upper side of the housing 500 of the adapter 50. In a state shown in FIGS. 9 and 10 in which the connector 46A is connected to the adapter 50, a shape of a portion of the adapter 50 and the first cord part 46 is equivalent to the connector 16A of the reusable endoscope 10 shown in FIG. 3. Therefore, the single-use endoscope 40 can be used with the same operation feeling as in a case of using the reusable endoscope 10, and the value of the single-use endoscope 40 can be increased.

In a case where the pair of interlocking members 463 are pressed in a state where the connector 46A is connected to the adapter 50, in conjunction with this, the front ends 465A of the pair of engagement members 465 retreat from the engagement recess 464, and the engagement between the recess 50Ba and the front ends 465A is released. In this state, the front ends 465A retreat to a position at which the front ends 465A do not come into contact with the restricting protrusion 50Bb. Therefore, in a state where the pair of interlocking members 463 are pressed, the connector 46A is movable in the rear direction Rr, and the connector 46A can be removed from the adapter 50. As described above, the engagement member 465 constitutes an engagement portion that is engageable with the adapter 50, and the interlocking member 463 constitutes a release portion that releases the engagement between the adapter 50 and the engagement member 465.

A mechanism for engaging the second adapter case 502 with the connector 46A is not limited to the mechanism shown in the drawings. For example, a configuration in which a recess is formed on the third surface 502C and a protrusion that engages with the recess is provided on the housing 460 of the connector 46A may be adopted. According to the configuration in which the engagement protrusion 50B is provided on the adapter 50 and the engagement recess 464 is provided on the connector 46A, an internal space of the housing 500 can be increased, and a space in a case of making the adapter 50 multifunctional can be secured.

An area of the third surface 502C is larger than an area of the first surface 502A and an area of the second surface 502B. As a result, a size of the engagement protrusion 50B (second connection portion) can be sufficiently increased, and the engagement with the connector 46A can be reliably performed. In addition, the connector 46A can be easily connected to the adapter 50. In addition, since the area of the third surface 502C is large, a distance between the first surface 502A and the second surface 502B in the front-rear direction can be secured, and the circuit board 50S having a large area can be disposed between the first surface 502A and the second surface 502B.

As shown in FIGS. 6 and 8, the secondary water supply fitting 462A is provided to protrude in the right direction R on a right-side surface of the housing 460 of the connector 46A. The secondary water supply fitting 462A is connected to the water supply pipeline 462 inside the housing 460. The air/water supply connection fitting 461A is provided to protrude in the left direction L on a left side surface of the housing 460 of the connector 46A. The air/water supply connection fitting 461A is connected to the air/water supply pipeline 461 inside the housing 460.

As shown in FIGS. 9 and 10, in a state where the connector 46A is connected to the adapter 50, the secondary water supply fitting 462A, which is connected to the air/water supply pipeline 461 and the water supply pipeline 462, and the air/water supply connection fitting 461A are exposed to the outside, are visible, and are contactable. That is, the air/water supply pipeline 461 and the water supply pipeline 462 are not inserted into the housing 500 of the adapter 50 and are configured not to communicate with an interior of the housing 500.

Both the air/water supply pipeline 461 and the water supply pipeline 462 communicate with the inside of the subject and allow the liquid to pass through. In the adapter 50, these pipelines are not inserted into the housing 500 that accommodates the substrate connector 50C. Therefore, the adapter 50 can be reliably separated from moisture to protect the electronic components. In addition, there is no concern that the adapter 50 will be contaminated by the liquid. Therefore, the adapter 50 can be repeatedly used instead of being used once. In a case of the single-use endoscope 40 as compared with a configuration in which the connector 46A and the adapter 50 are integrated, the manufacturing cost of the single-use endoscope 40 can be reduced by reusing the adapter 50. In addition, since the adapter 50 can have a watertight structure, the adapter 50 is configured to be reprocessed in the same manner as the reusable endoscope 10.

In addition, the single-use endoscope 40 can be used by being connected to a portable terminal equipped with an endoscope control device including a processor for the single-use endoscope 40, instead of the control device 80. In a case where the single-use endoscope 40 is connected to the portable terminal, the connector 47A is connected to the connector of the portable terminal, and the single-use endoscope 40 and the endoscope control device are electrically connected. In a case where the air/water supply pipeline 461 and the water supply pipeline 462 are used, the air/water supply device or a water supply cylinder is connected to the connector 46A. In this case, in a case where the connector 46A and the adapter 50 are integrated, the single-use endoscope 40 is difficult to handle due to the volume of the adapter 50. By separating the adapter 50 and the connector 46A from each other, in a case where the single-use endoscope 40 is connected to the portable terminal and used, the single-use endoscope 40 can be easily handled, and the efficiency of the endoscopy can be improved.

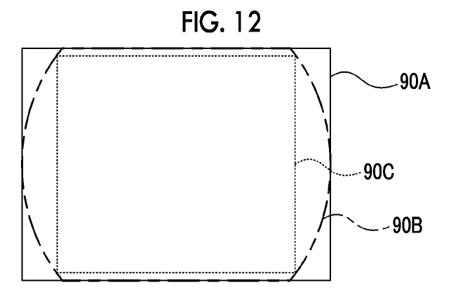

FIG. 11 is a block diagram showing an internal configuration in a state where the adapter 50 to which the connector 47A is connected is connected to the control device 80. As shown in FIG. 11, the housing 500 of the adapter 50 is provided with a power reception unit 51 that has the same function as the power reception unit 30 and that receives power wirelessly from the control device 80, a power supply generation unit 59 that has the same function as the power supply generation unit 29, an image signal transmission unit 58 that has the same function as the image signal transmission unit 28, an image signal modulation unit 57 that has the same function as the image signal modulation unit 27, a signal transmission and reception unit 55 that has the same function as the signal transmission and reception unit 25, a signal conversion unit 54 that has the same function as the signal conversion unit 24, an adapter controller 53 that controls each unit of the adapter 50 and the single-use endoscope 40, and a light source driving unit 56. The power supply voltage generated by the power supply generation unit 59 is supplied to each unit in the adapter 50 that requires power and each unit (including the imaging element 442 and the light source 443) in the single-use endoscope 40 that requires power.

In the state shown in FIG. 11, the light source driving unit 56 is electrically connected to the light source 443 by the signal line 471 provided in the second cord part 47. The adapter controller 53 is electrically connected to the imaging element 442 by the signal line 471 provided in the second cord part 47.

The light source driving unit 56 is a drive circuit that drives the light-emitting element included in the light source 443. The light source driving unit 56 drives the light source 443 in response to a command from the adapter controller 53 and emits light having a predetermined amount of light from the light source 443. By keeping the amount of light emitted from the light source 443 constant, the manufacturing cost of the single-use endoscope 40 can be reduced. In addition, by providing the light source driving unit 56 in the adapter 50, the manufacturing cost of the single-use endoscope 40 can be reduced.

The image signal transmission connector 531 shown in FIG. 9 is used for aligning the image signal transmission unit 58 of the adapter 50 and the image signal reception unit 83 of the control device 80. In particular, the image signal transmission unit 58 is disposed in an extension direction of a central axis of the image signal transmission connector 531. Light passes through the window W4 at the distal end of the image signal transmission connector 531, and transmission and reception of light based on the image signal is performed between the image signal transmission unit 58 and the image signal reception unit 83.

Transmission and reception of light based on the control signal is performed between the signal transmission and reception unit 55 and the signal transmission and reception unit 86 through the window W3 of the adapter 50.

The power reception unit 51 is disposed inside the first adapter case 501 at a position close to the connection surface of the first adapter case 501 with the connector connection portion 80A. A region of the surface 501S facing the power reception unit 51 constitutes the power reception region 51A. Since the power reception unit 51 is disposed inside the first adapter case 501, the power reception unit 51 is not exposed to the outside. The power supply voltage is generated from the power received by the power reception unit 51 close to the power reception region 51A, and the power supply voltage is supplied to the imaging element 442 or the light source 443 via the second cord part 47. Therefore, the power reception region 51A is a region for supplying power to the second cord part 47 connected to the substrate connector 50C.

The image signal modulation unit 57 and the signal conversion unit 54 constitute a conversion unit that converts the electric signal input from the second cord part 47 via the substrate connector 50C into an optical signal. The image signal transmission unit 58 and the signal transmission and reception unit 55 function as an optical transmission unit that transmits the optical signal to the control device 80.

The adapter 50 transmits and receives power wirelessly to and from the control device 80 and transmits and receives signals to and from the control device 80 by optical communication, as in the connector 16A. Therefore, the basic processing of the control device 80, such as the transmission of the power and the transmission and reception of the signals, can be made common to a case where the reusable endoscope 10 is used and a case where the single-use endoscope 40 is used, and the construction cost of the endoscope device 100 can be reduced with minimum modification of the program of the existing control device 80.

Next, the processing contents of the system controller 88 and the adapter controller 53 in a case where the single-use endoscope 40 is connected to the control device 80 via the adapter 50 by connecting the adapter 50 to which the connector 47A is connected to the connector connection portion 80A will be described.

In a case where the single-use endoscope 40 is used, the user operates the input device 80B to input a setting instruction of a second target value, which is a target value of the brightness of the image signal obtained by imaging with the single-use endoscope 40, to the system controller 88. The system controller 88 performs control of setting the target value of the brightness of the image signal to the second target value in response to the setting instruction, and transmitting target value information related to the second target value to the adapter 50.

The adapter controller 53 controls the light source driving unit 56 such that the amount of light emitted from the light source 443 is a predetermined constant value, without depending on the command from the system controller 88.

The adapter controller 53 controls the imaging element 442 in response to the command from the system controller 88. The adapter controller 53 controls at least one of the exposure time or the imaging sensitivity of the imaging element 442 based on the target value information related to the second target value transmitted from the system controller 88 and on the image signal output from the imaging element 442.

Specifically, the adapter controller 53 determines the exposure time and the imaging sensitivity such that the brightness of the image signal acquired from the imaging element 442 converges to the second target value by comparing the brightness with the second target value, and controls the imaging element 442 to perform imaging at the determined exposure time and imaging sensitivity.

As described above, the adapter controller 53 controls the exposure (brightness of the image signal output from the imaging element 442) of the imaging element 442 by changing at least one of the exposure time or the imaging sensitivity, instead of the amount of illumination light. The imaging sensitivity of the imaging element 442 is determined by an amplification rate (digital gain) of the signal amplification processing performed on the image signal by the adapter controller 53.

The adapter controller 53 may perform at least one of the white balance adjustment processing or the defect correction processing on the image signal output from the imaging element 442. As a result, the modification cost of the program of the control device 80 can be reduced.

In a form in which the adapter controller 53 performs the white balance adjustment processing and the defect correction processing, the system controller 88 generates the endoscopic image by performing necessary processing (demosaicing processing, gamma-correction processing, and the like) other than the white balance adjustment processing and the defect correction processing on the image signal received from the adapter 50. Then, the system controller 88 performs control of displaying the endoscopic image on the display device 90.

In the defect correction processing, it is necessary to acquire information on the defective pixel in the imaging element 442 in a case of manufacturing and to store the information in the memory of the single-use endoscope 40. Therefore, in order to reduce the manufacturing cost of the single-use endoscope 40, the defect correction processing may not be performed in both the adapter controller 53 and the system controller 88.

In a case where the defect correction processing is not performed as described above, in a situation in which the single-use endoscope 40 is outside the subject or the like, in a case where the imaging sensitivity of the imaging element 442 is set to an upper limit, the level of the pixel signal output from the defective pixel is greatly amplified, and the defect may be conspicuous in the endoscopic image obtained by imaging with the single-use endoscope 40.

Therefore, in a preparation stage (a state where the single-use endoscope 40 is suspended on a scope hanger or the like) before the single-use endoscope 40 is inserted into the subject, the adapter controller 53 sets an upper limit of the target value of the brightness of the image signal acquired from the single-use endoscope 40 to a first upper limit value. As a result, even in a case where the second target value set by the user is larger than the first upper limit value, since the target value is limited to the first upper limit value, the imaging sensitivity is also limited. In a case where it is determined that the single-use endoscope 40 is inserted into the subject, the adapter controller 53 sets the upper limit of the target value of the brightness of the image signal acquired from the single-use endoscope 40 to a second upper limit value higher than the first upper limit value.

As a result, it is possible to prevent the imaging sensitivity from being excessively high before the single-use endoscope 40 is inserted into the subject. As a result, the defect is less conspicuous in the endoscopic image obtained by imaging with the single-use endoscope 40, and it is possible to avoid a situation in which the operator determines that a failure has occurred before the start of the examination. On the other hand, in a state where the single-use endoscope 40 is inserted into the subject, the imaging sensitivity can be sufficiently increased in accordance with the target value of the brightness set by the operator, and the necessary observation performance can be maintained.

For example, in a case where there is no change in the endoscopic image captured by the single-use endoscope 40 (in a case where there is no change in the brightness of the image signal), the adapter controller 53 determines that it is before the insertion of the single-use endoscope 40, and in a case where a change has occurred in the endoscopic image (in a case where the brightness of the image signal is equal to or higher than a threshold value and the brightness is changed in a direction of being brighter), the adapter controller 53 determines that it is after the insertion of the single-use endoscope 40 based on a determination that the distal end part 44 is brought close to subject.

The imaging sensitivity of the imaging element 442 is changed by changing the digital gain multiplied by the image signal, but may be changed by changing the analog gain multiplied by the analog signal inside the imaging element 442. With the above-described control, in a case where the upper limit value of the analog gain is lowered before the insertion of the single-use endoscope 40, an effect of suppressing the heat generation of the distal end part 44 of the single-use endoscope 40 before the insertion of the subject can also be obtained.

The adapter controller 53 may not execute the structural emphasis processing performed on the endoscopic image before the insertion and execute the structural emphasis processing after the insertion, in addition to the change in the target value of the brightness of the image signal. As a result, the defect is less conspicuous in the endoscopic image, and it is possible to avoid a situation in which the operator determines that a failure has occurred before the start of the examination.

In a case where the single-use endoscope 40 is connected to the control device 80 via the adapter 50, it is preferable that the system controller 88 limits the function of the device included in the control device 80. Examples of the device in which the function is limited include the light source device 89. In a case where the single-use endoscope 40 is connected to the control device 80 via the adapter 50, the system controller 88 controls the amount of illumination light emitted from the light source device 89 to be equal to or less than the threshold value (for example, the minimum value that is configurable) or stops the light source device 89 such that the illumination light is not emitted. As a result, the power consumption of the control device 80 can be reduced. In a case where the amount of illumination light is set to the minimum value, there is an advantage in that the modification of the program of the control device 80 can be reduced as compared with a case where the light source device 89 is stopped.

Examples of the device in which the function is limited include the air supply device 85. In a case where the single-use endoscope 40 is connected to the control device 80 via the adapter 50, the system controller 88 stops the air supply device 85. As a result, the power consumption of the control device 80 can be reduced.

In a case where the single-use endoscope 40 is connected to the control device 80 via the adapter 50, the system controller 88 may limit the operation mode. For example, in a case where the single-use endoscope 40 is connected to the control device 80 via the adapter 50, the system controller 88 sets at least one of the light amount limiting mode or the transmission mode to be unconfigurable. For example, even in a case where the user performs a setting operation of enabling the light amount limiting mode or the transmission mode, the system controller 88 performs control of outputting an error sound or displaying an error message. Since the single-use endoscope 40 is equipped with a light-emitting element having a small amount of light at the distal end part 44, it is not a problem that the light amount limiting mode is unconfigurable. In addition, since it is difficult to transmit the illumination light from the light source 443 from the inside of the subject to the outside of the subject, it is preferable that the transmission mode is unconfigurable.

In a case where the endoscopic image (referred to as a first image) obtained by imaging with the reusable endoscope 10 and the endoscopic image (referred to as a second image) obtained by imaging with the single-use endoscope 40 are displayed, the system controller 88 preferably sets the display shape of the first image and the display shape of the second image to be different from each other.

FIG. 12 is a schematic diagram for describing an image displayed on the display device 90. FIG. 12 shows an endoscopic image 90A generated based on the image signal output from the endoscope. The system controller 88 performs control of applying an elliptical first mask 90B to the endoscopic image 90A and cutting out an elliptical image in the first mask 90B as the first image and displaying the first image on the display device 90. The system controller 88 performs control of applying a rectangular (in the example of the drawing, a square shape) second mask 90C to the endoscopic image 90A and cutting out a rectangular image in the second mask 90C as the second image and displaying the second image on the display device 90.

The second mask 90C is inscribed in the first mask 90B and is smaller than the first mask 90B. Therefore, a display area of the first image is larger than a display area of the second image. In the example of FIG. 12, the length of the second mask 90C in the up-down direction is slightly shorter than the length of the first mask 90B in the up-down direction. Therefore, it is preferable that the system controller 88 performs processing of enlarging the image in the second mask 90C in the endoscopic image 90A such that the length of the first image and the length of the second image in the up-down direction are the same.

As described above, by setting the display shapes of the first image and the second image to be different from each other, the operator can easily understand which of the reusable endoscope 10 or the single-use endoscope 40 is used. By reducing the size of the second image, the chip size of the imaging element 442 can be reduced, and the manufacturing cost of the single-use endoscope 40 can be reduced.

A program for controlling the adapter 50 and the single-use endoscope 40 is stored in the memory of the adapter controller 53 of the adapter 50. In addition, log information (the number of times of use of the adapter 50) of the adapter 50 is stored in the memory. The system controller 88 is configured to communicate with the adapter 50 even in a case where the adapter 50 is connected and the single-use endoscope 40 is not connected to the adapter 50. In the control device 80, a maintenance mode is configurable. In a case where the adapter 50 is connected to the control device 80, the maintenance mode is configurable. In a case where the maintenance mode is set, the system controller 88 acquires the log information from the adapter 50 or updates the program of the adapter 50. In a case where the maintenance of the adapter 50 is required, the maintenance can be executed only by connecting the adapter 50 to the control device 80 and setting the maintenance mode without connecting the single-use endoscope 40.

In a case where the single-use endoscope 40 is connected via the adapter 50, the system controller 88 may set a special light observation mode to be unconfigurable. It is preferable that the light source 443 provided at the distal end of the single-use endoscope 40 is not capable of generating a plurality of types of illumination light in order to reduce the manufacturing cost. Therefore, in a case where the single-use endoscope 40 is connected, by setting the special light observation mode to be unconfigurable, it is possible to prevent the setting of a mode that cannot be used.

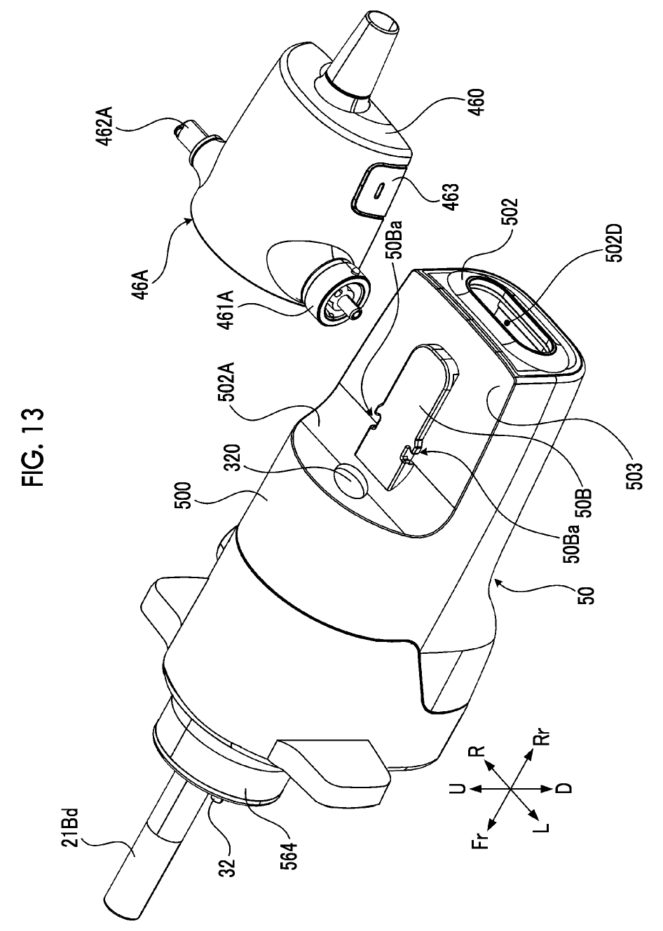

FIG. 13 is a perspective view showing a modification example of the adapter 50. The adapter 50 shown in FIG. 13 has a configuration in which the dummy fitting 32d is replaced with the air supply fitting 32 of the reusable endoscope 10, and a gas outlet 320 that allows the gas flowing into the air supply fitting 32 from the control device 80 to flow out is added to the first surface 502A of the housing 500.

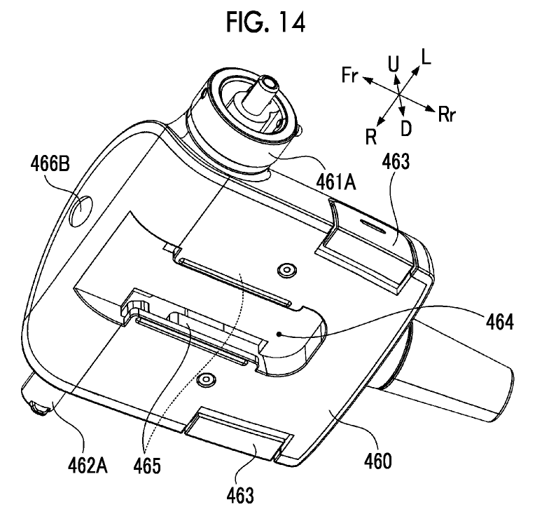

FIG. 14 is a perspective view showing a configuration example of the connector 46A connected to the adapter 50 shown in FIG. 13. The connector 46A shown in FIG. 14 has a configuration in which a gas inlet 466B that allows the gas flowing out from the gas outlet 320 to flow in is added to a surface facing the gas outlet 320 in a case where the connector 46A is connected to the adapter 50.

In a state where the adapter 50 is connected to the control device 80 and the connector 46A is connected to the adapter 50, the gas discharge port of the air supply device 85 inside the control device 80 and the gas inlet 466B of the connector 46A communicate with each other. The air/water supply pipeline 461 shown in FIG. 11 is connected to the gas inlet 466B. In this modification example, the air/water supply connection fitting 461A is replaced with the water supply connection fitting, and the water supply tank can be connected to the water supply connection fitting. The air discharged from the air supply device 85 flows into the air/water supply pipeline 461 through the adapter 50 and is discharged from the first discharge port 44D at the distal end of the single-use endoscope 40. According to this modification example, as in the reusable endoscope 10, the pressurized air can be sent into the water supply tank.

In the modification examples shown in FIGS. 13 and 14, a path through which the air from the air supply device 85 passes is provided inside the housing 500 of the adapter 50. However, since this air is clean, there is no concern that the adapter 50 will be contaminated. According to this modification example, since the air supply device 85 provided in the control device 80 can be used, the convenience can be improved.

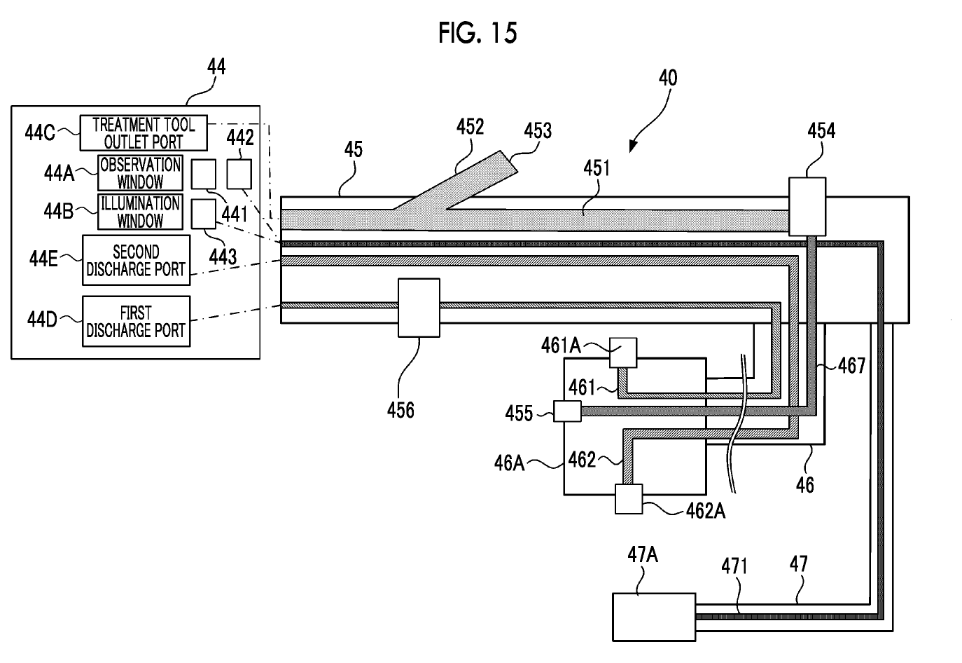

FIG. 15 is a schematic diagram showing a modification example of the single-use endoscope 40. The single-use endoscope 40 shown in FIG. 15 is different from the configuration shown in FIG. 5 in that the suction nozzle 455 provided in the operation unit 45 is provided in the connector 46A, and a suction pipeline 467 that extends from the valve mechanism 454 provided in the treatment tool pipeline 451 to the suction nozzle 455 of the connector 46A is added to the first cord part 46.

By connecting the suction device to the suction nozzle 455 and operating the valve mechanism 454, the substance inside the subject can be suctioned from the treatment tool outlet port 44C and can be discharged to the suction device through the treatment tool pipeline 451 and the suction pipeline 467. The suction nozzle 455 is configured to be visible or contactable in a state where the connector 46A is connected to the adapter 50. In a case where the connector 46A is connected to the adapter 50, the suction pipeline 467 is not able to communicate with the interior of the housing 500 of the adapter 50. Therefore, there is no concern that the adapter 50 will be contaminated by the body fluid or the like passing through the suction pipeline 467.

In the above description, the adapter 50 and the connector 46A are configured to be separated from each other, but the adapter 50 and the connector 46A may be integrated.

As described above, at least the following matters are described in the present specification. The following description in parentheses describes the corresponding components and the like in the above-described embodiment, but the present invention is not limited thereto.

(1) An adapter (adapter 50) comprising:
a first connection portion (first connection portion 50A) that is attachably and detachably connected to a control device (control device 80) that controls an endoscope;
a second connection portion (engagement protrusion 50B) to which a first cord part (first cord part 46) is attachably and detachably connected, the first cord part being provided to extend from an operation unit (operation unit 45) of the endoscope and including a liquid pipeline (air/water supply pipeline 461 or water supply pipeline 462) that communicates with a liquid discharge port (first discharge port 44D or second discharge port 44E) provided on a distal end side of the endoscope; and
a housing (housing 500) that is provided with the first connection portion and the second connection portion, the housing (housing 500) including a first surface (first surface 502A) that is on a side opposite to a first connection portion side in a movement direction (front-rear direction) of the housing in a case where the first connection portion is connected to the control device and that intersects the movement direction, a second surface (second surface 502B) that is on a side opposite to the first connection portion side with respect to the first surface in the movement direction and that intersects the movement direction, and a third surface (third surface 502C) that connects the first surface and the second surface,
in which the second connection portion is provided on the third surface.

(2) The adapter according to (1), further comprising:
a substrate (circuit board 50S) that is provided inside the housing between a position of the first surface and a position of the second surface in the movement direction.

(3) The adapter according to (1) or (2), further comprising:
a third connection portion (opening 502D and substrate connector 50C) that is provided on the second surface of the housing and to which a second cord part (second cord part 47) that is provided to extend from the operation unit and includes a signal line (signal line 471) is attachably and detachably connected.

(4) The adapter according to any one of (1) to (3),
in which the first surface and the second surface are provided at positions shifted in a first direction (up-down direction) as viewed in the movement direction.

(5) The adapter according to (4),
in which the third surface intersects the first direction.

(6) The adapter according to any one of (1) to (5),
in which an area of the third surface is larger than an area of the first surface and an area of the second surface.

(7) The adapter according to any one of (1) to (6),
in which the second connection portion is provided to protrude from the third surface.

(8) The adapter according to any one of (1) to (7),
in which the second connection portion is configured to be attached to and detached from the first cord part by moving in the movement direction of the first cord part.

### EXPLANATION OF REFERENCES

10: reusable endoscope
11, 41: insertion part
12, 42: soft part
13, 43: bendable part
14, 44: distal end part
14A, 44A: observation window
14B, 44B: illumination window
14C, 44C: treatment tool outlet port
14D: discharge port
15, 45: operation unit
16: universal cord
16A, 46A, 47A: connector
21: light guide
21A: optical fiber bundle
21B: light guide rod
21Bd: dummy rod
23: endoscope controller
24, 54, 87: signal conversion unit
25, 55, 86: signal transmission and reception unit
27, 57: image signal modulation unit
28, 58: image signal transmission unit
29, 59: power supply generation unit
30, 51: power reception unit
31, 531: image signal transmission connector
32: air supply fitting
32d: dummy fitting
33: water supply connection fitting
35: ventilation connector
40: single-use endoscope
44D: first discharge port
44E: second discharge port
46: first cord part
47: second cord part
50: adapter
50A: first connection portion
50B, 164, 564: engagement protrusion
50C: substrate connector
50S: circuit board
50Ba: recess
50Bb: restricting protrusion
51A: power reception region
53: adapter controller
56: light source driving unit
80: control device
80A: connector connection portion
80B: input device
81: power supply unit
82: power supply circuit
83: image signal reception unit
84: image signal demodulation unit
85: air supply device
88: system controller
89: light source device
90: display device
90A: endoscopic image
90B: first mask
90C: second mask
91: power supply
100: endoscope device
141, 441: optical system
142, 442: imaging element
161: first connector case 1
62: second connector case
163: third connector case
320: gas outlet
443: light source
451: treatment tool pipeline
452: branch path
453: treatment tool inlet port
454, 456: valve mechanism
455: suction nozzle
460, 500, 800: housing
461: air/water supply pipeline
461A: air/water supply connection fitting
462: water supply pipeline
462A: secondary water supply fitting
463: interlocking member
464: engagement recess
465: engagement member
465A: front end
466B: gas inlet
467: suction pipeline
471: signal line
501: first adapter case
501S: surface
502: second adapter case
502A: first surface
502B: second surface
502C: third surface
502D: opening
801: second engagement recess
802, 806: window
803: first engagement recess
804: insertion hole
805: air supply hole
810: power supply region
SL1: signal line
W1, W2, W3, W4: window

What is claimed is:

1. An adapter comprising:
a first connection portion that is attachably and detachably connected to a control device that controls an endoscope;
a second connection portion to which a first cord part is attachably and detachably connected, the first cord part being provided to extend from an operation unit of the endoscope and including a liquid pipe line that communicates with a liquid discharge port provided on a distal end side of the endoscope; and
a housing that is provided with the first connection portion and the second connection portion, the housing including a first surface that is on a side opposite to a first connection portion side in a movement direction of the housing in a case where the first connection portion is connected to the control device and that intersects the movement direction, a second surface that is on a side opposite to the first connection portion side with respect to the first surface in the movement direction and that intersects the movement direction, and a third surface that connects the first surface and the second surface,
wherein the second connection portion is provided on the third surface.

2. The adapter according to claim 1, further comprising:
a substrate that is provided inside the housing between a position of the first surface and a position of the second surface in the movement direction.

3. The adapter according to claim 1, further comprising:
a third connection portion that is provided on the second surface of the housing and to which a second cord part that is provided to extend from the operation unit and includes a signal line is attachably and detachably connected.

4. The adapter according to claim 1,
wherein the first surface and the second surface are provided at positions shifted in a first direction as viewed in the movement direction.

5. The adapter according to claim 2,
wherein the first surface and the second surface are provided at positions shifted in a first direction as viewed in the movement direction.

6. The adapter according to claim 3,
wherein the first surface and the second surface are provided at positions shifted in a first direction as viewed in the movement direction.

7. The adapter according to claim 4,
wherein the third surface intersects the first direction.

8. The adapter according to claim 5,
wherein the third surface intersects the first direction.

9. The adapter according to claim 6,
wherein the third surface intersects the first direction.

10. The adapter according to claim 1,
wherein an area of the third surface is larger than each of an area of the first surface and an area of the second surface.

11. The adapter according to claim 1,
wherein the second connection portion is provided to protrude from the third surface.

12. The adapter according to claim 1,
wherein the second connection portion is configured to be attached to and detached from the first cord part by moving in the movement direction of the first cord part.
